(19) United States
(12) Patent Application Publication
(10) Pub. No.: US 2026/0165869 A1
(43) Pub. Date: Jun. 18, 2026

(54) EXTERNAL NASAL DILATOR WITH A RESILIENT MEMBER DISCONNECTED FROM THE BASE LAYER

(71) Applicant: ASO LLC, SARASOTA, FL (US)

(72) Inventor: Edmund A. Sinda, Bradenton, FL (US)

(21) Appl. No.: 19/413,038

(22) Filed: Dec. 9, 2025

Related U.S. Application Data
(60) Provisional application No. 63/733,013, filed on Dec. 12, 2024.

(51) Int. Cl. A61F 5/08

(52) U.S. Cl. CPC A61F 5/08

(57) ABSTRACT

An external nasal dilator includes a base layer adhereable to the human nose, a resilient member extending laterally across the base layer, and a cover layer over the resilient member and the base layer. Relief cuts are positioned on opposed lateral sides of the external nasal dilator and longitudinally sever the cover layer along terminal edges of the resilient member. The relief cuts do not extend into the base layer. When the external nasal dilator is attached to the human nose, the relief cuts form opposed raised flaps extending out of the base layer, which are disconnected from the base layer.

### CROSS-REFERENCE TO RELATED APPLICATION

This claims the benefit of priority from provisional Application No. 63/733,013, filed Dec. 12, 2024, which is incorporated by reference in its entirety.

### FIELD

This relates to the field of nasal dilators and, more particularly, to external nasal dilators.

### BACKGROUND

External nasal dilators are worn on the bridge of the nose and gently dilate the nasal passages. Such dilators are composed of a resilient strip of material attached to an adhesive substrate. When the dilator is adhered to the skin of the nose, the resilient strip acts as a spring, applying an outward force to the nose during inhalation.

Some conventional external nasal dilators have so called “relief cuts” that are formed by cutting through every layer of the external nasal dilator, including the resilient strip. When worn on the nose, the relief cuts form raised flaps composed of a portion of the cover layer, a portion of the resilient strip, and a portion of the base layer that adheres to the nose. The raised flaps create holes extending through the external nasal dilator because all three layers are adhered together.

### BRIEF SUMMARY

Nasal dilators with relief cuts may be improved by eliminating the hole through the base layer, which will increase the surface area of the portion of the base layer that adheres to the nose. This provides better adhesion to the nose. They may also be improved by not completely adhering the resilient member to the base layer in the area surrounding the relief cuts. This allows the raised flaps to be disconnected from the base layer. The external nasal dilators described here include one or more of these improvements.

A first example of such an external nasal dilator is configured to apply a dilating force to laterally opposed nasal valves of a human nose. The external nasal dilator comprises a base layer having a back surface and a resilient member engaging surface opposite the back surface. The back surface includes adhesive for adhering the base layer to the human nose. A cover layer is attached to the base layer. A resilient member is between the base layer and the cover layer and applies the dilating force to the laterally opposed nasal valves when the base layer is adhered to the human nose. The resilient member extends laterally across the resilient member engaging surface from a left terminal edge of the resilient member to a right terminal edge of the resilient member. A left relief cut longitudinally severs the cover layer along the left terminal edge, forming a left opening through the cover layer which makes a raised left flap extending out of the cover layer when the base layer is adhered to the human nose. A right relief cut longitudinally severs the cover layer along the right terminal edge, forming a right opening through the cover layer which makes a raised right flap extending out of the cover layer when the base layer is adhered to the human nose. The left relief cut and the right relief cut do not extend into the base layer. The raised right flap and the raised left flap are disconnected from the base layer.

The external nasal dilator may include one or more of the following additional features.

The left relief cut and the right relief cut may completely longitudinally sever the resilient member.

The left relief cut and the right relief cut may longitudinally sever the cover layer and the resilient member.

The resilient member may be directly adhered to the cover layer and not directly adhered to the base layer.

The resilient member may not be adhered to the base layer with adhesive.

An adhesive-free portion of the resilient member engaging surface may directly contact the resilient member from the left terminal edge to the right terminal edge.

A second example of such an external nasal dilator is configured to apply a dilating force to laterally opposed nasal valves of a human nose. The external nasal dilator comprises a base layer having a back surface and a resilient member engaging surface opposite the back surface. The base layer is laterally elongated. The back surface includes adhesive for adhering the base layer to the human nose. A cover layer is laminated to the base layer. The cover layer and base layer collectively define an outer perimeter of the external nasal dilator. A resilient member is between the base layer and the cover layer and applies the dilating force to the laterally opposed nasal valves when the base layer is adhered to the human nose. The resilient member extends laterally across the resilient member engaging surface from a left terminal edge of the resilient member to a right terminal edge of the resilient member. The resilient member is configured to generate 5 grams to 35 grams of dilating force. A left relief cut longitudinally severs the cover layer along the left terminal edge, forming a left opening through the cover layer which makes a raised left flap extending out of the cover layer when the base layer is adhered to the human nose. A right relief cut longitudinally severs the cover layer along the right terminal edge, forming a right opening through the cover layer which makes a raised right flap extending out of the cover layer when the base layer is adhered to the human nose. The left relief cut and the right relief cut do not extend into the base layer. The resilient member is directly adhered to the cover layer and is not directly adhered to the base layer.

The external nasal dilator may include one or more of the following additional features.

The left relief cut and the right relief cut may completely longitudinally sever the resilient member.

The left relief cut and the right relief cut may longitudinally sever the cover layer and the resilient member.

The resilient member may not be adhered to the base layer with adhesive.

An adhesive-free portion of the resilient member engaging surface may directly contact the resilient member from the left terminal edge to the right terminal edge.

An example of a method of making such an external nasal dilator comprises obtaining an external nasal dilator configured to apply a dilating force to laterally opposed nasal valves of a human nose via adhesive attachment of the external nasal dilator to the human nose. The external nasal dilator comprises (a) a base layer adhereable to the human nose, (b) a resilient member extending laterally across the base layer and which applies the dilating force when the external nasal dilator is adhered to the human nose, and (c) a cover layer over the resilient member and the base layer. The method includes forming a left relief cut in the external nasal dilator by longitudinally kiss cutting the cover layer along a left terminal edge of the resilient member without cutting into the base layer, thereby creating a left opening through the cover layer. The method includes forming a right relief cut laterally opposite the left relief cut by longitudinally kiss cutting the cover layer along a right terminal edge of the resilient member without cutting into the base layer, thereby creating a right opening through the cover layer.

The method may include one or more of the following additional features.

Kiss cutting may be achieved by making a cut through the cover layer and the resilient member without cutting into the base layer.

The left relief cut and the right relief cut may completely longitudinally sever the resilient member.

The left relief cut and the right relief cut may longitudinally sever the cover layer and the resilient member.

The resilient member may be directly adhered to the cover layer and not directly adhered to the base layer.

The resilient member may not be adhered to the base layer with adhesive.

An adhesive-free portion of the resilient member engaging surface may directly contact the resilient member from the left terminal edge to the right terminal edge.

The left opening may make a raised left flap extending out of the cover layer when the base layer is adhered to the human nose. The right opening may make a raised right flap extending out of the cover layer when the base layer is adhered to the human nose. The raised right flap and the raised left flap are disconnected from the base layer.

### BRIEF DESCRIPTION OF THE DRAWINGS

FIG. 1 is a front view of an example of an external nasal dilator.

FIG. 2 is a back view of the external nasal dilator of FIG. 1.

FIG. 3 is a front perspective exploded view of the external nasal dilator of FIG. 1.

FIG. 4 is a back perspective exploded view of the external nasal dilator of FIG. 1.

FIG. 5 is a top view of the external nasal dilator of FIG. 1 with the bottom view being a mirror image of the top view.

FIG. 6 is a right side view of the external nasal dilator of FIG. 1 with the left side view being a mirror image of the right side view.

FIG. 7 is a cross section of the external nasal dilator of FIG. 1 along plane 7-7 of FIG. 5.

FIG. 8 is a front perspective view the external nasal dilator of FIG. 1 slightly bent to induce separation in the relief cuts.

FIG. 9 is a close up view of the external nasal dilator of FIG. 8.

FIG. 10 is a side view of the external nasal dilator of FIG. 1 being worn on a person's nose in use.

FIG. 11 is a top view of the external nasal dilator of FIG. 1, illustrating a technique for forming relief cuts.

FIG. 12 is a top view of a second example of an external nasal dilator.

FIG. 13 is a top view of a third example of an external nasal dilator.

### DETAILED DESCRIPTION OF EXAMPLES

This disclosure describes certain examples, but not all examples of the external nasal dilator and related methods. Where a particular feature is disclosed in the context of a particular example, that feature can also be used, to the extent possible, in combination with and/or in the context of other examples. The external nasal dilator and related methods may be embodied in many different forms and should not be construed as limited to only the features and examples described here.

Compared to conventional external nasal dilators with relief cuts, the external nasal dilators described here have more adhesive surface area for adhesion to the nose. This is because the conventional external nasal dilators have holes through the base layer created by the relief cuts. In the external nasal dilators described here, the relief cuts are present yet the holes are eliminated. The external nasal dilators described here also require less adhesive because some or all portions of the resilient member do not need to be directly adhered to the base layer.

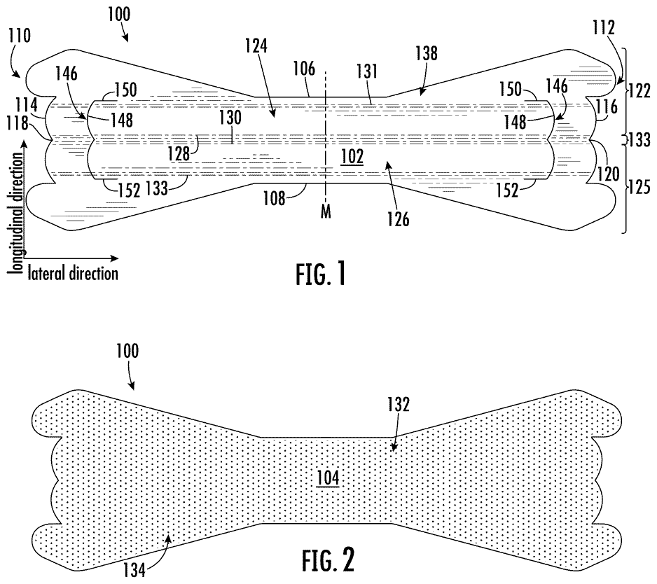

Referring to FIGS. 1 and 2, an example of the external nasal dilator 100 includes a front surface 102, back surface 104 opposite the front surface 102, top edge 106, bottom edge 108 opposite the top edge 106, left end 110, and right end 112 opposite the left end 110. The external nasal dilator 100 extends in a lateral direction from the left end 110 to the right end 112 and longitudinally from the top edge 106 to the bottom edge 108. The external nasal dilator 100 is substantially symmetric about an imaginary longitudinal midline M.

The top edge 106 defines the top perimeter of the external nasal dilator 100 and extends from the left end 110 to the right end 112. In use, the wearer will place the external nasal dilator 100 across the bridge of the nose evenly about the midline M. When worn properly, the midline M is substantially coaxial with an anatomical midline of the nose.

The top edge 106 has a concave curvature between the left end 110 and right end 112 such that the top edge 106 curves downwardly from the left end 110 and right end 112 approaching the midline M.

The bottom edge 108 defines a bottom perimeter of the external nasal dilator 100 and extends from the left end 110 to the right end 112. Between the left end 110 and right end 112, the bottom edge 108 has a concave curvature such that the bottom edge 108 curves upwardly approaching the midline M from the left end 110 and right end 112, respectively.

The left end 110 includes a left edge 114, defining a left perimeter of the external nasal dilator 100. The right end 112 includes a right edge 116, defining a right perimeter of the external nasal dilator 100. The left edge 114 and right edge 116 join with the top edge 106 and bottom edge 108 to collectively define the entire outer perimeter of the external nasal dilator 100.

The left edge 114 and right edge 116 converge toward the midline M to a left convergence point 118 and a right convergence point 120, respectively. The region above the left convergence point 118 and right convergence point 120 to the top edge 106 is an upper section 122 of the external nasal dilator 100. The region below the left convergence point 118 and right convergence point 120 to the bottom edge 108 is a lower section 125 of the external nasal dilator 100.

This example of the external nasal dilator 100 includes two resilient members: an upper resilient member 124 and a lower resilient member 126.

The upper resilient member 124 is carried by the upper section 122. The upper resilient member 124 is positioned on the external nasal dilator 100 in such a way that the upper resilient member 124 extends laterally across the upper section 122 from the left edge 114 to the right edge 116 and applies a dilating force to the nasal valves when the external nasal dilator 100 is attached to the human nose.

The lower resilient member 126 is carried by the lower section 125 of the external nasal dilator 100 and is positioned on the external nasal dilator 100 in such a way that the lower resilient member 126 extends laterally across the lower section 125 from the left edge 114 to the right edge 116 and applies a dilating force to the nasal valves when the external nasal dilator 100 is attached to the human nose.

In this example, the external nasal dilator 100 has two distinct resilient members 124, 126, but other examples of the external nasal dilator 100 may have a single resilient member or more than two resilient members. In every example, the resilient member(s) need not extend straight between the left edge 114 and right edge 116 as shown. In other examples, the resilient member(s) may be curved.

In the example shown, the longitudinal width of the upper resilient member 124 and the lower resilient member 126 are substantially equal. The longitudinal width of the upper resilient member 124 and the lower resilient member 126 are not required to be equal in every example.

An intermediate section 133 extends longitudinally between a bottom edge 128 of the upper resilient member 124 and a top edge 130 of the lower resilient member 126 and laterally between the left edge 114 and right edge 116. The convergence points 118, 120 are within the intermediate section 133.

In other examples, the left edge 114 and right edge 116 converge outwardly away from the midline M to form a region that protrudes outwardly from the midline M rather than forming a recess as shown. In such examples, the convergence points 118, 120 are located at respective terminal ends of the protrusions.

The back surface 104 forms a nose contacting surface that contacts the skin of the nose when worn and adheres the external nasal dilator 100 thereto using a skin contact adhesive 132, which is represented by dotted shading. In certain examples, the skin contact adhesive 132 covers substantially the entire the back surface 104. In other examples, the skin contact adhesive 132 may cover only portions of the back surface 104.

The skin contact adhesive 132 is an adhesive suitable for skin contact. Skin contact adhesives can adhere to the skin but are removable without substantially damaging the skin. Examples of skin contact adhesives include, but are not limited to, acrylic adhesives, silicone adhesives, hydrogels, hydrocolloids, silicone, or the like. The adhesive material may also include an additive that provides benefits to the skin such as vitamins, vitamin E, and/or zinc oxide, for example.

The back surface 104 may be covered by a release liner (not shown) that forms part of the external nasal dilator's 100 packaging and is designed to cover the skin contact adhesive 132 prior to use. Before the wearer applies the external nasal dilator 100 to the nose, the wearer removes the release liner to expose the skin contact adhesive 132 and then adheres the external nasal dilator 100 to the nose.

The release liner may have a single piece construction that covers the adhesive 132 or a two-piece construction with a seam proximal to the midline M of the external nasal dilator 100.

The release liner is typically a paper or plastic film used to prevent the skin contact adhesive 132 from prematurely adhering. The release liner may be composed of a base material that is coated on one or both sides with a release agent that facilitates easy removal from the adhesive 132. The release liner may be made of any conventional nasal dilator release liner material, such as polymer-coated paper, for example.

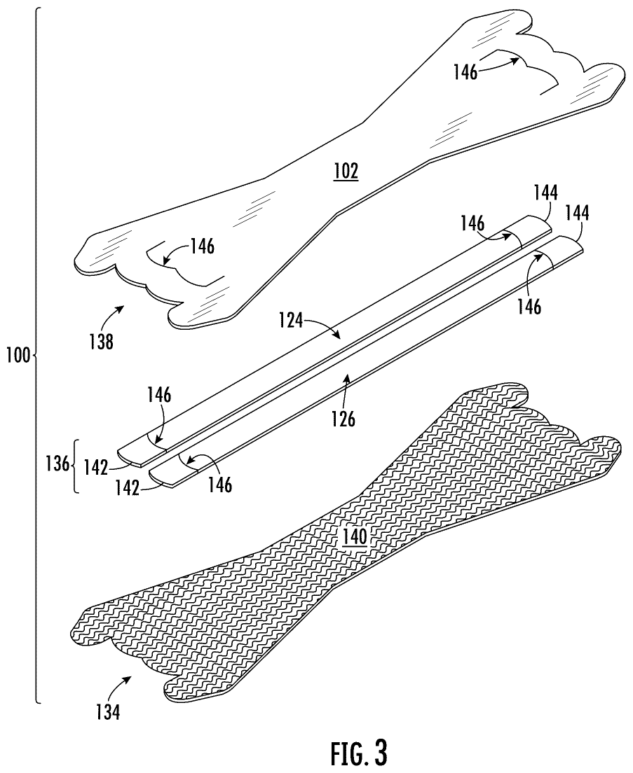

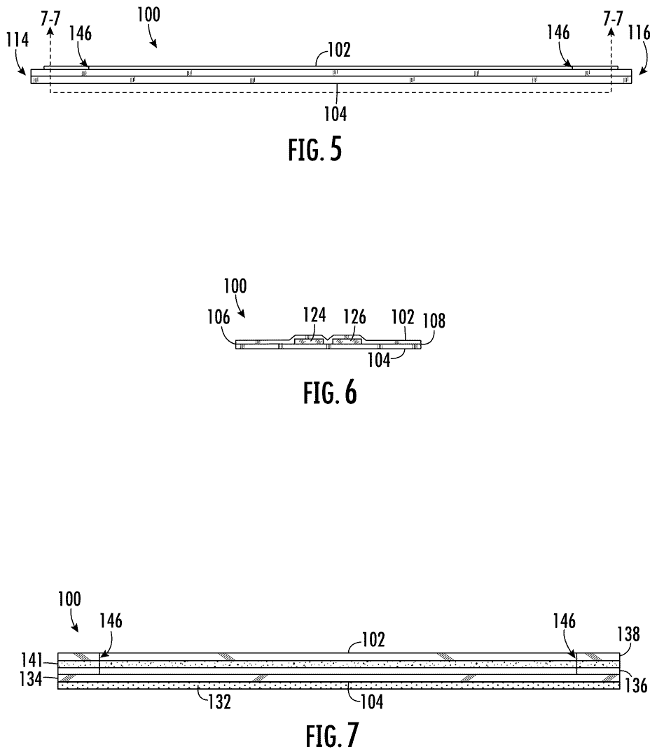

Referring to FIGS. 3-6, the external nasal dilator 100 is composed of a plurality of laminated layers including a base layer 134, resilient layer 136, and cover layer 138.

The base layer 134 and cover layer 138 may be composed of thin, flexible material that is comfortable. Such a material may include, for example, woven or non-woven fabric, such as polyester, polyethylene, polypropylene, polyurethane, or the like. In the alternative, the material may include a plastic woven or non-woven material, such as polyethylene, polypropylene, polyurethane, or the like. In other examples, the material may be a foam, silicone, or the like.

The base layer 134 includes the back surface 104 having the skin contact adhesive 132 thereon for adhering the external nasal dilator 100 to the nose. The base layer 134 also includes a resilient member engaging surface 140 opposite the back surface 104, which is adapted to contact the resilient layer 136.

In some particular examples, the base layer 134 has a thickness of about 0.5 to about 500 μm. The base layer 134 may be oxygen and/or moisture permeable or impermeable, depending on the materials chosen.

The resilient layer 136 includes at least one resilient member. In the example shown, the resilient layer 136 includes the upper resilient member 124 and the lower resilient member 126. In other examples, the resilient layer 136 has one resilient member or more that two resilient members.

The resilient member(s) of the resilient layer 136, such as the upper resilient member 124 and lower resilient member 126 may extend completely to the left edge 114 and right edge 116 or may terminate proximal to, but short of, the left edge 114 and right edge 116.

In the example shown, the upper resilient member 124 and lower resilient member 126 extend substantially parallel in the lateral direction of the external nasal dilator 100. The upper resilient member 124 and lower resilient member 126, respectively, include a first end 142 and a second end 144.

Resilient members such as the upper resilient member 124 and lower resilient member 126 may be made from any suitable material having the desired flexibility and resiliency to generate the desired amount of dilating force, such as metal, plastic, or the like. The dilating force is the spring biasing force created by the resiliency of the resilient members 124, 126. The resilient members 124, 126 are typically flat, semi-rigid, and resilient so as to generate a dilating force when flexed across the nose, which is what lifts the exterior wall tissues of the nostrils. This dilating force is typically between about 5 grams and 35 grams, 15 and 35 grams, or sometimes about 25 grams. In a particular example, the resilient members 124, 126 are made of biaxially oriented polyester with a thickness of 0.1 mm to 0.3 mm and a longitudinal width of 3 mm to 6 mm. The longitudinal width and/or front to back thickness of the resilient members 124, 126 may be selected to obtain an external nasal dilator 100 with the desired amount of dilating force. Other particular examples of materials from which a resilient member 124, 126 can be made include polyethylene, polypropylene, or the like.

The dilating force strength of resilient members may be measured by conventional techniques. U.S. Pat. No. 11,344,444 provides such an example in which the resilient member is compressed into U-shape while a scale measures the force.

The number and design of the resilient members of the resilient layer 136 may depend on the desired amount of force to be applied to the nose, the direction of force to be applied to the nose, and aesthetic considerations.

The cover layer 138 is primarily made of a thin, flexible material. The cover layer 138 may be made of the same material as the base layer 134 or a different material from the base layer 134. The cover layer 138 and base layer 134 may be laminated together using an adhesive, such as a thermally fusable adhesive or the like. The cover layer 138 may have a thickness of about 1 to about 500 μm and may be oxygen and moisture permeable or impermeable. The cover layer 138 may also include a woven textile and/or nonwoven textile. The cover layer 138 may also be colored to match different skin tones or have decorative colors. The cover layer 138 may also include decorative features and or indicia such as words, logos and the like printed thereon or attached thereto.

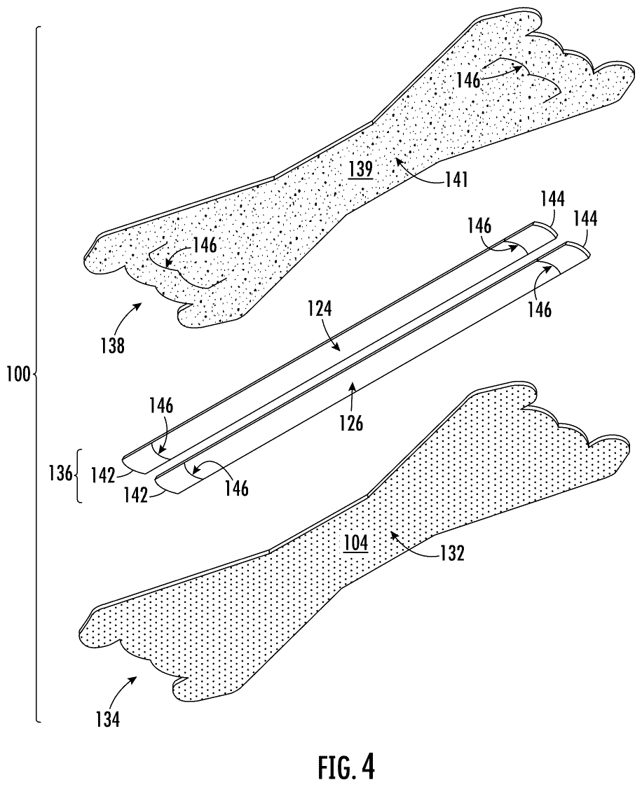

The cover layer 138 includes a bottom surface 139 including a cover layer adhesive 141 thereon, which is illustrated by the dotted shading in FIG. 4 on the bottom surface 139. The cover layer adhesive 141 adheres the cover layer 138 to the resilient layer 136 and to the resilient member engaging surface 140 of the base layer 134 when these components are laminated together.

Referring to FIGS. 1-5 and 7, the external nasal dilator 100 includes a pair of opposed relief cuts 146 that extend completely through the cover layer 138 and resilient layer 136. The relief cuts 146 do not extend through the base layer 134. As best shown in FIGS. 3, 4, and 7. The relief cuts 146 longitudinally sever the material making up the cover layer 138 and resilient layer 136. The relief cuts 146 are positioned at laterally opposed ends of the external nasal dilator 100.

The relief cuts 146 include an outside edge 148, an upper cut edge 150, and a lower cut edge 152. The outside edge 148 defines a longitudinal width of the cut 146. The upper cut edge 150 and lower cut edge 152 extend laterally toward the midline M from the outside edge 148. The curvature of the outside edge 148 may mimic the portion of the left edge 114 or right edge 116 laterally adjacent to the cut 146.

Along the outside edge 148, the upper resilient member 124 and lower resilient member 126 are severed. The upper cut edge 150 and lower cut edge 152 extend inward toward the midline M substantially parallel to a top edge 131 of the upper resilient member 124 and a bottom edge 133 of the lower resilient member 126.

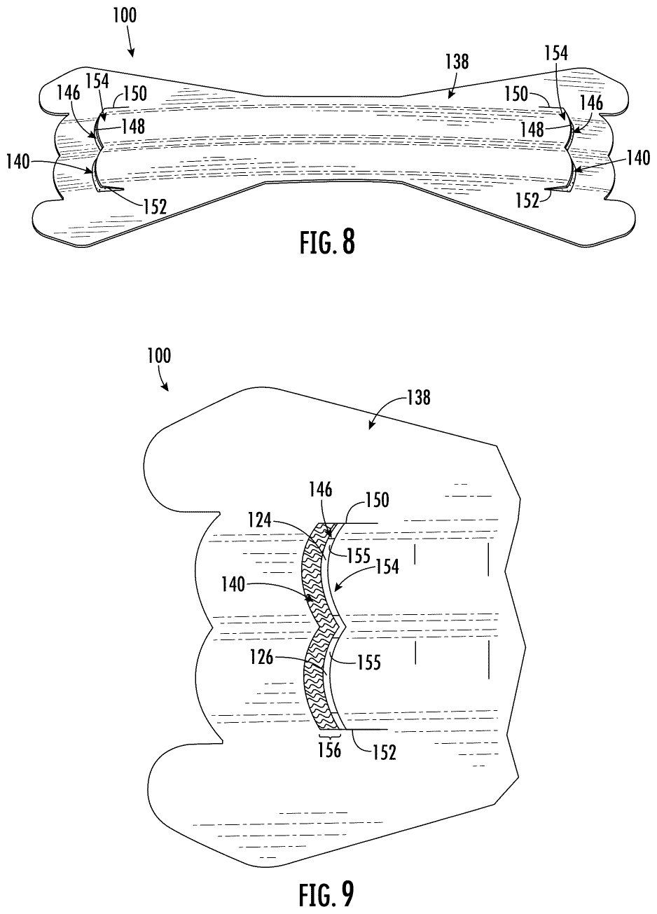

Referring to FIGS. 8 and 9, the external nasal dilator 100 is slightly bent to illustrate the relief cuts 146 in more detail. When adhered to and positioned across the bridge of a wearer's nose, a material separation caused by the cuts 146 forms opposed raised flaps 154, leaving a gap 156 caused by the separation. The length of the flaps 154 determines the degree of separation. As best seen in FIGS. 8 and 9 the relief cuts 146 longitudinally sever the cover layer 138 along a first terminal edge 155 of the resilient layer 136 and its resilient members 124, 126 thereby forming an opening through the cover layer 138.

In FIG. 3, resilient member engaging surface 140 is shaded with wavy lines. In FIGS. 8 and 9, the wavy lines of the resilient member engaging surface 140 are visible through the gaps 156. This is because the relief cuts 146 do not extend through the base layer 134, which means the base layer 134 does not form part of the flaps 154.

The area of the resilient members 124, 126 around the relief cuts 146 are not adhered directly to the resilient member engaging surface 140. This permits the raised flaps 154 to be disconnected from the resilient member engaging surface 140. In some examples, the resilient members 124, 126 are not adhered directly to the resilient member engaging surface 140 at all. In other examples the resilient members 124, 126 may be adhered directly to the resilient member engaging surface 140, but not in the vicinity of the relief cuts 146, such as along the midline M, for example. In each example, the resilient member engaging surface 140 has an adhesive-free portion thereof that directly contacts the resilient member 124, 126.

Accordingly, the resilient layer 136 and its resilient members 124, 126 are held in place by being adhered to the bottom surface 139 of the cover layer 138. When the cover layer 138, resilient layer 136, and base layer 134 are laminated together as in FIGS. 1, 2, 5, 6, and 7 either the portion of the resilient member engaging surface 140 directly in contact with the resilient layer 136 does not include adhesive or the resilient layer 136 including its one or more resilient members 124, 126 are adhered to the resilient member engaging surface 140, but not under the relief cuts 146. This allows the resilient layer 136 to freely float over the resilient member engaging surface 140 when the cover layer 138, resilient layer 136, and base layer 134 are laminated together.

The external nasal dilator 100 described here does not have openings from the relief cuts 146 on the base layer 134 because the relief cuts 146 do not extend through the base layer 134. This gives the external nasal dilator 100 more adhesive surface area on the back surface 104 than a conventional nasal dilator with conventional relief cuts that extend through every layer.

Referring to FIG. 10, the external nasal dilator 100 is shown adhered to the exterior wall of a human nose. When adhered to the nose, the external nasal dilator 100 applies dilating forces F to the exterior wall, which lifts the exterior wall tissue of the nostrils and dilates the nasal valves. Dilating the nasal valves reduces airflow resistance and increases airflow through the nasal cavities when breathing. The right side of the nose is a mirror image of FIG. 10. The flaps 154 redirect the angle of the dilating force F applied by the resilient layer 136, changing it from primarily peel forces and tensile forces into primarily shear forces. This redirected dilating force F is imparted to the external wall of the nose.

In the example shown, the upper resilient member 124 and lower resilient member 126 each have an effective length responsible for creating the dilating force F. The effective length of a given resilient member is the lateral length from the first end 142 to the second end 144 thereof. When cut, the effective length of a resilient member decreases because the cuts 146 define the effective length of the resilient member. Because the cuts 146 reduce the amount of the lateral length of the resilient member that creates the dilating force F, this, in turn, allows a resilient member to generate more dilating force F than the uncut resilient member would otherwise be able to generate.

The external nasal dilator 100 may be used in a method of applying dilating force to a human nose. This method includes adhering the external nasal dilator 100 to the exterior wall of the nose in such a way that the external nasal dilator 100 is positioned symmetrically about the bridge of the nose.

The external nasal dilator 100 may be used in any situation in which the wearer desires to stabilize and or expand the nasal cavities, such as when ill, during exercise, while sleeping, and many other uses.

An example of a method of making such an external nasal dilator includes forming a first relief cut in the external nasal dilator by longitudinally cutting the cover layer along a first terminal edge of the resilient member without cutting into the base layer, thereby creating a first opening through the cover layer. The method also includes forming a second relief cut positioned on a second lateral side of the external nasal dilator opposite the first relief cut by longitudinally cutting the cover layer along a second terminal edge of the resilient member without cutting into the base layer, thereby creating a second opening through the cover layer. The first opening and second opening through the cover layer correspond to the openings shown in FIGS. 8 and 9 under the raised flaps 154.

Examples of methods that are used to manufacture conventional external nasal dilators are described in U.S. Pat. Nos. 8,062,329; 8,858,587; and 8,641,852.

The external nasal dilator may be manufactured by die cutting them from a continuous laminate of the material layers of FIG. 7 or the individual layer components may be die cut individually from one or more continuous material layers before or during assembly of the completed laminate.

In the method, the relief cuts 146 may be formed using a cutting method such as a blade that carefully cuts through the cover layer 138 and resilient layer 136, but without cutting through the base layer 134.

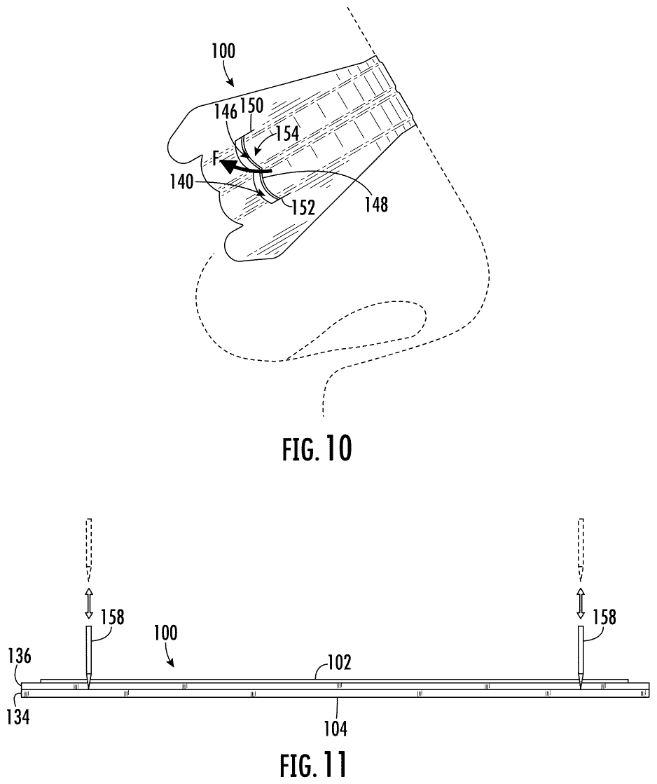

A particular example of such a cutting method is kiss cutting, which is illustrated in FIG. 11. In this cutting method, an external nasal dilator 100 without relief cuts is passed under one or more blades 158 that move up and down, forming the relief cuts 146 without cutting through the base layer 134.

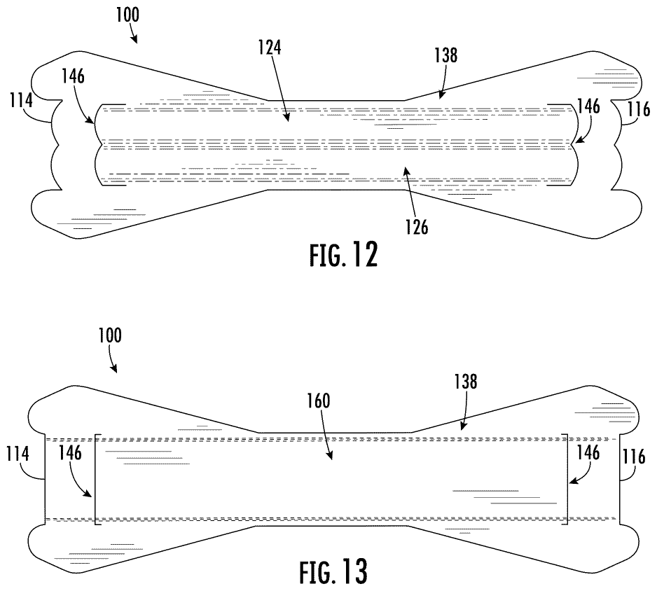

The design of the external nasal dilator 100 shown in FIGS. 1-10 is one of many possible designs and is described in detail by way of example only. FIGS. 12 and 13 show two additional examples, from numerous possible examples, of the external nasal dilator 100.

The external nasal dilator 100 of FIG. 12 is similar to that of FIG. 1 except that the upper resilient member 124 and lower resilient member 126 do not extend from the left edge 114 to the right edge 116. Instead, the upper resilient member 124 and lower resilient member 126 terminate on the left and right sides at a respective relief cut 146.

The external nasal dilator 100 of FIG. 13 includes a single resilient member 160 that extends from the left edge 114 to the right edge 116.

The external nasal dilators may be packaged for sale by placing each dilator in an individual wrapper and placing the wrapped dilators into a box, bag, or the like for consumers to purchase. Packages of multiple dilators may contain dilators of the same dilating force strength or dilators of different dilating strengths. In some examples, the wrappers may form an array of dilators having different dilating force strengths. In other examples, the wrappers and/or dilators themselves may include a color or other indicia thereon that indicates the relative dilating force strength of the dilator.

The external nasal dilator and related methods are not limited to the details and features described in connection with the example embodiments. There are numerous variations and modifications of the nasal dilator and methods that may be made without departing from the scope of what is claimed.

That which is claimed is:

1. An external nasal dilator configured to apply a dilating force to laterally opposed nasal valves of a human nose, the external nasal dilator comprising:
a base layer having a back surface and a resilient member engaging surface opposite the back surface, the back surface including adhesive for adhering the base layer to the human nose;
a cover layer attached to the base layer;
a resilient member between the base layer and the cover layer and which applies the dilating force to the laterally opposed nasal valves when the base layer is adhered to the human nose, the resilient member extending laterally across the resilient member engaging surface from a left terminal edge of the resilient member to a right terminal edge of the resilient member;
a left relief cut longitudinally severing the cover layer along the left terminal edge, forming a left opening through the cover layer which makes a raised left flap extending out of the cover layer when the base layer is adhered to the human nose; and
a right relief cut longitudinally severing the cover layer along the right terminal edge, forming a right opening through the cover layer which makes a raised right flap extending out of the cover layer when the base layer is adhered to the human nose;
wherein (a) the left relief cut and the right relief cut do not extend into the base layer and (b) the raised right flap and the raised left flap are disconnected from the base layer.

2. The external nasal dilator of claim 1, wherein the left relief cut and the right relief cut completely longitudinally sever the resilient member.

3. The external nasal dilator of claim 1, wherein the left relief cut and the right relief cut longitudinally sever the cover layer and the resilient member.

4. The external nasal dilator of claim 1, wherein the resilient member is directly adhered to the cover layer and is not directly adhered to the base layer.

5. The external nasal dilator of claim 1, wherein the resilient member is not adhered to the base layer with adhesive.

6. The external nasal dilator of claim 1, wherein an adhesive-free portion of the resilient member engaging surface directly contacts the resilient member from the left terminal edge to the right terminal edge.

7. An external nasal dilator configured to apply a dilating force to laterally opposed nasal valves of a human nose, the external nasal dilator comprising:
a base layer having a back surface and a resilient member engaging surface opposite the back surface, the base layer being laterally elongated, the back surface including adhesive for adhering the base layer to the human nose and;
a cover layer laminated to the base layer, the cover layer and base layer collectively defining an outer perimeter of the external nasal dilator;
a resilient member between the base layer and the cover layer and which applies the dilating force to the laterally opposed nasal valves when the base layer is adhered to the human nose, the resilient member extending laterally across the resilient member engaging surface from a left terminal edge of the resilient member to a right terminal edge of the resilient member, the resilient member being configured to generate 5 grams to 35 grams of dilating force;
a left relief cut longitudinally severing the cover layer along the left terminal edge, forming a left opening through the cover layer which makes a raised left flap extending out of the cover layer when the base layer is adhered to the human nose; and
a right relief cut longitudinally severing the cover layer along the right terminal edge, forming a right opening through the cover layer which makes a raised right flap extending out of the cover layer when the base layer is adhered to the human nose;
wherein (a) the left relief cut and the right relief cut do not extend into the base layer and (b) the resilient member is directly adhered to the cover layer and is not directly adhered to the base layer.

8. The external nasal dilator of claim 7, wherein the left relief cut and the right relief cut completely longitudinally sever the resilient member.

9. The external nasal dilator of claim 7, wherein the left relief cut and the right relief cut longitudinally sever the cover layer and the resilient member.

10. The external nasal dilator of claim 7, wherein the resilient member is not adhered to the base layer with adhesive.

11. The external nasal dilator of claim 7, wherein an adhesive-free portion of the resilient member engaging surface directly contacts the resilient member from the left terminal edge to the right terminal edge.

12. A method comprising:
obtaining an external nasal dilator configured to apply a dilating force to laterally opposed nasal valves of a human nose via adhesive attachment of the external nasal dilator to the human nose,
the external nasal dilator comprising (a) a base layer adhereable to the human nose, (b) a resilient member extending laterally across a resilient member engaging surface of the base layer and which applies the dilating force when the external nasal dilator is adhered to the human nose, and (c) a cover layer over the resilient member and the base layer;
forming a left relief cut in the external nasal dilator by longitudinally kiss cutting the cover layer along a left terminal edge of the resilient member without cutting into the base layer, thereby creating a left opening through the cover layer; and
forming a right relief cut laterally opposite the left relief cut by longitudinally kiss cutting the cover layer along a right terminal edge of the resilient member without cutting into the base layer, thereby creating a right opening through the cover layer.

13. The method of claim 12, wherein kiss cutting is achieved by cutting through the cover layer and the resilient member without cutting into the base layer.

14. The method of claim 12, wherein the left relief cut and the right relief cut completely longitudinally sever the resilient member.

15. The method of claim 12, wherein the left relief cut and the right relief cut longitudinally sever the cover layer and the resilient member.

16. The method of claim 12, wherein the resilient member is directly adhered to the cover layer and is not directly adhered to the base layer.

17. The method of claim 12, wherein the resilient member is not adhered to the base layer with adhesive.

18. The method of claim 12, wherein an adhesive-free portion of the resilient member from the left terminal edge to the right terminal edge.

19. The method of claim 12, wherein:
the left opening makes a raised left flap extending out of the cover layer when the base layer is adhered to the human nose;
the right opening makes a raised right flap extending out of the cover layer when the base layer is adhered to the human nose; and
the raised right flap and the raised left flap are disconnected from the base layer.
